(19) United States
(12) Patent Application Publication
(10) Pub. No.: US 2019/0015038 A1
(43) Pub. Date: Jan. 17, 2019

(54) IN VIVO PATIENT COMPLIANCE MONITORING

(71) Applicant: TruTag Technologies, Inc., Kapolei, HI (US)

(72) Inventor: Hod Finkelstein, Berkeley, CA (US)

(21) Appl. No.: 16/036,853

(22) Filed: Jul. 16, 2018

Related U.S. Application Data
(60) Provisional application No. 62/533,186, filed on Jul. 17, 2017.
(60) Provisional application No. 62/554,887, filed on Sep. 6, 2017.
(60) Provisional application No. 62/563,401, filed on Sep. 26, 2017.

(51) Int. Cl. A61B 5/00; G01J 1/46; G01J 1/02; G01N 21/64; A61B 5/07; A61B 1/04; A61K 49/00

(52) U.S. Cl. CPC A61B 5/4833; G01J 1/46; G01J 1/0238; G01N 21/643; A61B 5/0084; G01J 2001/442; A61B 5/7282; A61B 5/076; A61B 5/686; A61B 1/041; A61K 49/0017; A61B 5/0071

(57) ABSTRACT

An in vivo patient compliance apparatus includes a timing controller, a pulsed light source, and a detector. The timing controller controls the timing of the activation of the pulsed light source and the detector. The detector includes a single-photon avalanche diode (SPAD), a time integrator coupled to the SPAD, and an event counter coupled to the SPAD. The time integrator is configured to store charge in response to receiving a signal from the SPAD, and configured to stop storing charge in response to receiving a signal from the timing controller. The event counter is configured to store a preset amount of charge in response to receiving a signal from the SPAD.

### CROSS REFERENCE TO RELATED APPLICATIONS

This application claims the benefit of priority to U.S. Provisional Application No. 62/533,186, filed on Jul. 17, 2017; U.S. Provisional Application No. 62/554,887, filed on Sep. 6, 2017; and U.S. Provisional Application No. 62/563,401, filed on Sep. 26, 2017. All of the foregoing are incorporated herein by reference in their entirety for all purposes.

### BACKGROUND

### 1. Field of the Disclosure

The present disclosure relates to in vivo patient compliance monitoring and more specifically to an apparatus and process for detecting in vivo a fluorescent marker added to a patient's medication for detecting the presence of the medication inside the body of the patient.

### 2. Description of the Related Art

Patient noncompliance or nonadherence can be a threat to the patient's health and wellbeing. Patient noncompliance may be the result of many different factors including a patient's misunderstanding of a doctor's instructions, a patient forgetting to take a medication, or simply a patient ignoring a healthcare advice. Traditional ways of controlling patient compliance typically rely on indirect methods. For example, a patient may determine whether a dose of a specific medication has been taken by identifying whether the medication is present in a pill case. This can be ineffective as there could be other reasons why a particular medication is not present in a pill case. Another way of controlling patient compliance is self-reporting. For example, a smartphone application alerts a patient that a dose of a particular medication is due and the patient self-reports (e.g., by pressing a button) that the dose has been taken. This can also be ineffective as the patient may falsely self-report that the dose has been taken. These indirect methods further suffer from the deficiency that a patient may inadvertently consume the wrong dose of a medication or the wrong medication altogether.

### SUMMARY

Disclosed is an in vivo patient compliance apparatus and method for monitoring whether a certain medication has been taken and recording the times the medication has been taken. The in vivo patient compliance apparatus and method directly determines whether a particular medication has been taken by identifying whether the medication is present inside the body of the patient (e.g., in the bloodstream of the patient). In particular, the vivo patient compliance apparatus determines whether a specific fluorophore that was added to the medication is present in the body of the patient. In some embodiments, the vivo patient compliance apparatus is attached to a pacemaker or other device to be inserted to the body of the patient. The vivo patient compliance apparatus then wirelessly communicates with an external device for reporting whether the fluorophore was detected.

The in vivo patient compliance apparatus includes a timing controller, a pulsed light source, and a detector. The timing controller controls the timing of the activation of the pulsed light source and the detector. The detector includes a single-photon avalanche diode (SPAD), a time integrator coupled to the SPAD, and an event counter coupled to the SPAD. The time integrator is configured to store charge in response to receiving a signal from the SPAD, and configured to stop storing charge in response to receiving a signal from the timing controller. The event counter is configured to store a preset amount of charge in response to receiving a signal from the SPAD.

In some embodiments, the time integrator includes a first transistor controlled by the SPAD, a second transistor controlled by the timing controller, and a capacitor configured to store charge in response to the first and second transistors being turned on. Furthermore, in some embodiments, the event counter includes a pulse generator coupled to the SPAD, a first transistor controlled by the pulse generator, a second transistor controlled by the timing controller, and a capacitor configured to store charge in response to the first and second transistor being turned on.

The method for determining patient compliance includes initializing the time integrator and the event counter of the in vivo patient compliance apparatus. Then for each cycle of a sequence of cycles, a light pulse is generated by the light source of the in vivo patient compliance apparatus. The SPAD of the in vivo patient compliance apparatus is charged and the arrival of a photon is detected using the charged SPAD. If a photon is detected by the SPAD, the time integrator is started and the value of the counter is increased. Finally, after all the cycles of the sequence of cycles have been completed, the value of the time integrator is compared with the value of the event counter to identify the presence of a specific fluorophore. In some embodiments, comparing the values of the time integrator and the event counter involves determining a ratio of an output of the time integrator and an output of the event counter, and comparing the determined ratio to an expected value.

### BRIEF DESCRIPTION OF DRAWINGS

The disclosed embodiments have other advantages and features, which will be more readily apparent from the detailed description, the appended claims, and the accompanying figures (or drawings). A brief introduction of the figures is below.

FIG. 1A illustrates an environment of a system for in vivo patient compliance, according to one embodiment.

FIG. 1B illustrates a block diagram of a system for in vivo patient compliance integrated with a pacemaker or camera pill, according to one embodiment.

FIG. 2 illustrates a time diagram of the fluorescence emission of a fluorescent marker, according to one embodiment.

FIG. 3A illustrates a block diagram of a system for in vivo patient compliance, according to one embodiment.

FIG. 3B illustrates a block diagram of a system for in vivo patient compliance, according to another embodiment.

FIG. 4A illustrates a circuit diagram of the time integrator of FIG. 3A, according to one embodiment.

FIG. 4B illustrates a circuit diagram of the event counter of FIG. 3A, according to one embodiment.

FIG. 5A illustrates a flow diagram of a process for in vivo patient compliance determination, according to one embodiment.

FIG. 5B illustrates a flow diagram of a process for in vivo patient compliance determination, according to one embodiment.

FIG. 6 illustrates a block diagram of an environment of a system for in vivo patient compliance communicating with an external client device.

### DETAILED DESCRIPTION

The Figures (FIGS.) and the following description relate to preferred embodiments by way of illustration only. It should be noted that from the following discussion, alternative embodiments of the structures and methods disclosed herein will be readily recognized as viable alternatives that may be employed without departing from the principles of what is claimed.

Reference will now be made in detail to several embodiments, examples of which are illustrated in the accompanying figures. It is noted that wherever practicable similar or like reference numbers may be used in the figures and may indicate similar or like functionality. The figures depict embodiments of the disclosed system (or method) for purposes of illustration only. One skilled in the art will readily recognize from the following description that alternative embodiments of the structures and methods illustrated herein may be employed without departing from the principles described herein.

### Configuration Overview

In certain applications, such as for monitoring compliance of patients in taking medications, it is desirable to monitor whether a certain medication has been taken by a user and record the times it has been taken. Furthermore, it is desirable to do this in a way which causes the least disruption to the patient and which is cost effective. Passive schemes where no active transmission of data from the medication are also desirable. Furthermore, it is desirable that false detections that the medication has not been taken (false positives) and that missed detections of when the medication has in fact been taken (false negatives) be reduced.

Therefore, in some embodiments, provided herein is a system for monitoring patient compliance with a treatment regime using an implantable device capable of sensitive and specific detection of fluorophores in vivo. In some embodiments, provided herein is a system comprising an integrated circuit coupled to a pacemaker or a camera pill to detect the presence or absence of a population of fluorophores for patient compliance by detecting single photons emitted by fluorophores in vivo and verifying that the photons detected by the device are characteristic of those emitted by the fluorophore of interest. The system is thus capable of highly sensitive and specific detection of a plurality of fluorescent molecules at low concentrations in vivo. In preferred embodiments, the system also operates under a low power scheme.

The invention described here addresses these desirable features by adding a fluorescent marker to medications and using a detector to collect a signal from the marker. The fluorescent marker can be added to any type of medication, whether they be pills, tablets, capsules, liquid oral dose, liquid IV form, or any other format of administration. For the purposes of illustration, one example involves a 750 mg pill with 7.5 mg of an added fluorophore that is taken by the patient. Assuming all of the fluorophore molecules reach the blood stream of the patient, and that the total volume of blood is 5 liters for the patient, the initial concentration of the drug in the blood would be 1.5 mg/liter. A detector which collects an optical signal from 1 ml of blood would collect a signal from 1.5 micrograms of fluorophores. In an actual patient, one would expect that the actual amount of fluorophore accessible for imaging to be even smaller.

Fluorescence imaging has been used for identification of target molecules. In this context, fluorescence is the emission of a photon within an emission band by a fluorophore after excitation by absorption of a photon with a higher excitation energy. Fluorescence is typically characterized by a Stokes shift between the excitation and fluorescence wavelengths, and is also characterized by a typical fluorescence decay time, i.e., the fluorescence emission probability per unit time of each fluorophore, which is often characterized by a mono- or multi-exponential probability curve. More specifically, the fluorescence lifetime is the time after which l/e of excited electrons emit a photon and return to their ground state. Since the mean of a probability density function of an exponential distribution

$$f ( t ) = λ {e}^{- λ t} is \frac{1}{λ} ,$$

if one calculates the emission times of a sufficiently large number of photons from a population of electrons which have been excited at the same time, the mean arrival time of the photons with respect to the excitation time will be the fluorescence lifetime.

The current invention includes an in-vivo, ultra-low-power, highly-specific, non-imaging system for monitoring for the presence or absence of a specific fluorophore. In some embodiments, the system includes a pulsed light source that emits light including a spectrum of wavelengths that can be absorbed by and excite a plurality of fluorophores to induce fluorescence. In some embodiments, the pulsed light source includes a filter between the light source and the external environment to limit the emitted light from substantially overlapping with the fluorescence spectrum of the fluorophores.

The system also comprises a sensor for detecting photons emitted from excited fluorophores. In some embodiments, the sensor is capable of detecting the presence or absence of the fluorophore according to the known fluorescence lifetime of the fluorophore after excitation. This can be done by detecting photons in an environment for selected time intervals after a pulsed light has been emitted to determine whether there is a presence or absence of a photon emission decay associated with a fluorophore of interest (e.g., fluorophores bound to a drug or excipient to monitor patient compliance). In some embodiments, the system also comprises an emission filter between the sensor and the environment to limit photon detection to those within an emission spectrum specific to the fluorophore of interest and/or to block the excitation light.

In this manner, the system can be used to detect the presence or absence of a plurality of target fluorophores in a low signal/high background environment by discrimination based on the time of detection after emission of a pulse of light from a pulsed light source that includes an excitation spectrum for the fluorophore of interest. Further resolution can be achieved through the use of excitation and/or emission filters, as described. In some embodiments, the sensor is only configured to detect the presence of a photon emitted from a fluorophore, and does not detect spatial information about the source of the photon.

Without limiting the generality of the present disclosure, in one example, a detection device is mounted on an implantable, battery-powered device with an ability to transmit information outside the body to a receiver. In some embodiments, the detector is mounted on an internal device implanted within a patient. In some embodiments, the detector is mounted on a pacemaker.

In another example, a detection device is mounted on an ingestible, battery-powered device with an ability to transmit information outside the body to a receiver. In some embodiments, the detector is mounted on an internal device ingested by the patient. In some embodiments, the detector is mounted on a camera pill. In some embodiments, the camera pill is an ingestible pill including a camera system for imaging an area of interest, a power source, such as a battery. In some embodiments, the camera pill includes a wireless interface for communicating to an external device. In some embodiments, the detection device uses the power source of the camera pill. In some embodiments, the detection device uses the wireless interface of the camera pill to communicate detection information to an external device.

The detection device is designed to detect the presence of a fluorescent molecule which was incorporated in a medication. The fluorescent molecule may be incorporated in bulk, in solution or in a coating, or via other mechanisms of association/incorporation, and the fluorescent molecule is released into the body (e.g., the blood stream) after the medication is taken (e.g., ingested, etc.) by the patient. In some implementations, the fluorescent molecule may be released into the digestive tract, be absorbed into tissue, or be dispersed to other parts of the body.

In some embodiments the fluorescent molecule may be packaged such that it only comes in contact with bodily fluids in certain environments so that chemical modifications to the fluorophore are minimized and its functional life in the body is extended. In some embodiments, the fluorescent molecules are packaged in small compartments, which are transparent to at least excitation and fluorescence wavelengths and which compartments do not dissolve in blood, such that the fluorescent molecules are protected from damage by bodily fluids. In some embodiments, the fluorescent molecules are packaged in small compartments whose dissolution rate in the body is known a priori.

System Architecture

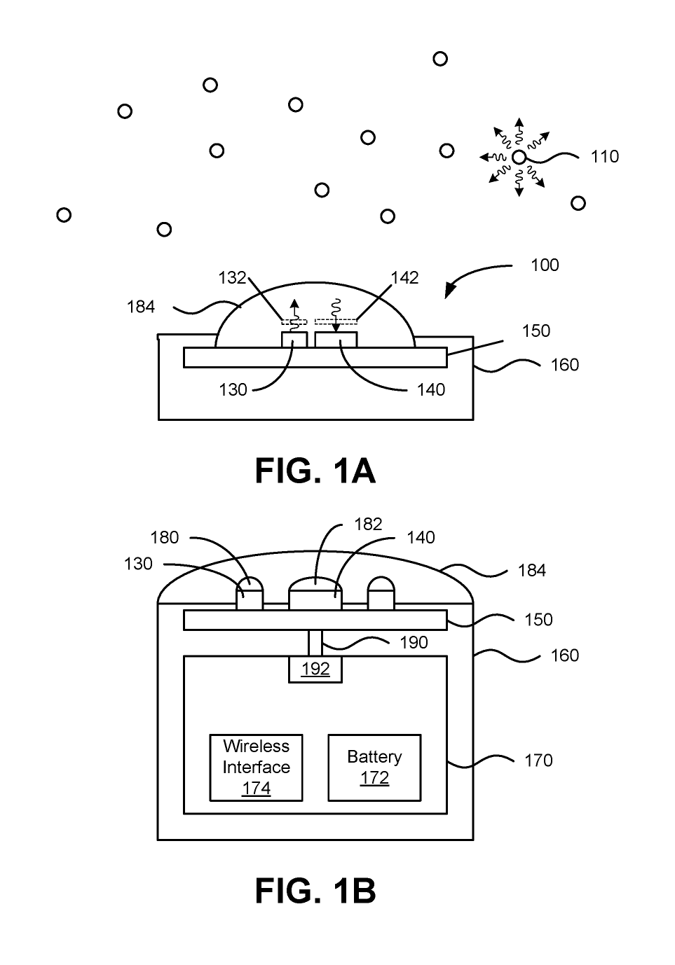

FIG. 1A illustrates an environment of a system for in vivo patient compliance, according to one embodiment.

FIG. 1A illustrates a sensor 100 surrounded by fluorophores 110, each having the same characteristic single or multi-exponential fluorescent decay probability curve. In some embodiments, each fluorophore 110 has a Stokes shift that allows removal of some or all of the excitation energy but not some or any of the fluorescence energy before reaching the detector, e.g., by filtration. In some embodiments, the Stokes shift is at least 50 nm. Thus, after excitation of a volume around the detector by a pulse of light including light in the excitation band of the fluorophores of interest, photons may be emitted by the plurality of excited fluorophores according to the fluorescence decay probability statistics specific to the fluorophore of interest. The light flux of the source is designed such that, at target concentrations, at most one photon will be detected by the sensor. In one embodiment, the probability of detection of a fluorescence photon in the presence of the target fluorophores is less than 1%. The sensor's response correlates with the time of arrival of the detected photon. Subsequently, other pulses of excitation light similarly illuminate the volume and subsequent fluorescent photon arrivals are correlated to the response of the sensor. After a sufficient number of photon arrival times are recorded, directly or indirectly, by the sensor, the distribution of the detected photon's arrival times with respect to the excitation time is likely to be consistent with the decay probability curve associated with the fluorophore of interest. Thus, photon detection consistent with the exponential decay/photon emission probability of the fluorophore of interest is used to identify the presence or absence of the fluorophores 110 in an in vivo system. In some embodiments, the fluorophore 110 has an excitation wavelength or a fluorescence wavelength that is heavily absorbed in the interrogated medium. Increasing the amount of absorption in the surrounding volume decreases the amount of scattering, which may affect the photons' time-of-arrival distribution. In another embodiment, the fluorophore 110 has an excitation wavelength or a fluorescence wavelength that is not heavily absorbed in the surrounding volume, but the effect of scattering on typical photon arrival times is known and is deconvolved from the measured arrival times so that the fluorescence lifetime can be extracted. This may interfere with the accuracy of detection of the fluorophore emission distribution over time, and thus detection of the fluorophore 110, as described below.

The sensor 100 includes a fluorescence detector 140. The sensor 100 also includes a pulsed light source 130. The fluorescence detector 140 and the pulsed light source 130 are both in electrical communication with a printed-circuit board (PCB) 150 (i.e., an electrical substrate and interconnect board). The printed-circuit board (PCB), pulsed light source 130, and fluorescence detector 140 can be isolated from the external environment by an enclosure 160. The PCB 150 contains driver circuitry, which causes the pulsed light source 130 to emit short pulses of excitation light. The fluorescence detector 140 detects fluorescence emitted by the fluorophores 110. In some embodiments, the pulsed light source 130, the fluorescence detector 140, and the PCB is enclosed in an enclosure 160 with a sufficiently transparent cap 184 to allow transmission of light from the pulsed light source 130 to the external environment and from a fluorophore 110 in the external environment to the fluorescence detector 140. In some embodiments, an excitation filter 132 is incorporated into the system along the path of light between the pulsed light source 130 and the external environment (suspected of comprising fluorophores 110). The excitation filter can reduce non-specific fluorescence by limiting the emission spectrum to within the wavelengths effective for excitation of a fluorophore of interest 110. In some embodiments, a fluorescence emission filter 142 is incorporated into the system, which is preferentially transmissive in all or a subset of the fluorescence emission band of the fluorophore and blocks light outside of the fluorescence emission band.

FIG. 1B a block diagram of a system for in vivo patient compliance integrated with an implantable device, such as a pacemaker or a camera pill, according to one embodiment. The detector 140 is capped by a lens 182 and is attached and electrically interconnected to a PCB 150. One or more pulsed light sources 130, which may also be capped by lenses 180 are also attached to the PCB 150. In some embodiments, the lens 180 ensures illumination of a large volume from the light sources 130. In some embodiments, the lens 182 ensures capture of light by the fluorescence detector 140 from approximately the same volume that is illuminated by the light sources 130. In some embodiments, the lens 180 or 182 has a large angular range (e.g., a fish eye lens). In some embodiments, the lens 180 or 182 has a 160 degree of acceptance angle and a 5 mm working distance. In some embodiments, the pulsed light source and the detector have similar focal points or focal regions, such that light emanating from the light source illuminates the same region which is imaged onto the detector.

In some embodiments, the lenses may be coated with a filter to reduce transmission of selected wavelengths of light and increase sensitivity and specificity for the fluorophore of interest. In some embodiments, the lens 180 covering the pulsed light source 130 is coated by a filter that reduces transmission of light outside of the excitation spectrum of the fluorophore of interest. In some embodiments, the pulsed light source 130 is coated by a filter that reduces transmission of light outside of the excitation spectrum of the fluorophore of interest. In some embodiments, the lens 182 covering the fluorescence detector 140 is coated by a filter that removes some or all of the excitation light but not some or any of the fluorescence light before reaching the detector. In some embodiments the sensor element is coated by a filter which removes some or all of the excitation light. In some embodiments, the filter for the detector 140 selectively reduces transmission of light outside of the emission spectrum of the fluorophore of interest.

In some embodiments, the PCB 150 includes additional components for operating the sensor 140 and pulsed light sources 130. The PCB 150 is electrically connected through an interconnect 190 to a receiver 192 on the pacemaker 170. In some embodiments, the PCB 150 is electrically connected through an interconnect 190 to a receiver 192 on a camera pill 170. In some embodiments, the PCB 150 is connected to the receiver 192 wirelessly. The implantable device 170 comprises a power source, such as a battery 172 and a wireless interface 174 for communicating to the outside world. In some embodiments, the whole device is enclosed in an enclosure 160 with a sufficiently transparent cap 184.

The one or more pulsed light sources 130 of the sensor 100 may be present in multiple configurations sufficient to illuminate the surrounding medium with short pulses of fluorophore excitation energy that is coordinated with detection of photons by the fluorescence detector 140 by the PCB 130. The pulsed light source 130 is configured to emit pulses of light including an energy/wavelength spectrum capable of excitation of the fluorescent molecules 110. Upon absorption of a photon, the fluorophores 110 will fluoresce stochastically with an emission probability governed by a characteristic fluorescence lifetime (e.g., a mono- or multi-exponential decay function). A portion of the light emitted by the fluorophores will then reach the detector 140 such that, for each light pulse, the probability of detecting a photon on an individual sensor element is low, for example less than 1%.

The sensor 100 may be implanted inside the body of a person. For example, the sensor 100 is included in a pacemaker implanted on the chest or abdomen of a patient or in another type of bodily implant. The sensor may be also implanted in the brain or other portions of the body. In some embodiments, the sensor 100 is included in a camera pill ingested by a patient.

The sensor 100 may draw power from a battery and may include wireless capabilities to communicate with a client device outside of the body of the patient. In some embodiments, the sensor 100 shares the battery or the wireless transceivers with other devices that are implanted inside the body of the patient. In some embodiments, the sensor 100 further includes an enclosure 160 to mechanically or chemically isolate the sensor 100 from the in-vivo environment.

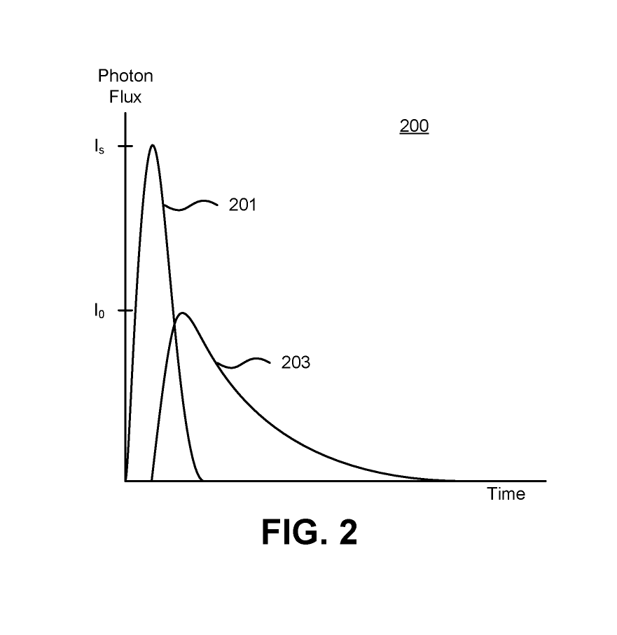

FIG. 2 qualitatively illustrates a time diagram of the fluorescence emission of a fluorescent marker, according to one embodiment. The vertical axis (i.e., the y-axis) of the time diagram 200 represents the photon flux of the pulsed light source or the fluorophores. The horizontal axis (i.e., the x-axis) of the time diagram 200 represents the time elapsed since the pulsed light source is activated.

As shown by the time diagram, after the pulsed light source is activated, the pulsed light source emits a pulse of light 201. In some embodiments, in order to allow the detector 140 to be able to distinguish between photons emitted by the pulsed light source 201 and photons emitted by the fluorophores 203, the pulsed light source is quickly turned on and off. More specifically, the pulsed light source is driven such that the light intensity of the pulsed light source is cutoff significantly faster than the decay rate of the light intensity of the light emitted by the fluorophores 110.

In some embodiments, the pulsed light source 201 has a rise time of 0.5 ns or less. In some embodiments, the pulsed light source 201 has a duration of 2 ns or less. In some embodiments, the pulsed light has a cycle period (time between subsequent pulses) of 1 μs or less. In some embodiments, the pulse duration is sufficiently shorter than the decay time constant of a fluorophore to allow detection of a sufficient amount of fluorophore emission for discrimination from background. In some embodiments, the pulse width and the cycle period are based on the decay time constant of the fluorophore detection to allow detection of a fluorophore in an in vivo environment.

Fluorophores emit light after excitation according to a known mono or multi-exponential decay curve. The decay curve governs the probability of an emission over time from an excited fluorophore. That is, the light intensity of the fluorescent emission of a plurality of fluorophores 110 according to a simple exponential decay behaves as follows:

I(t)=I₀e⁻t/τ (1)

Where I₀ is the peak light intensity of the fluorescence emission, t is the time elapsed from excitation, and τ is a time decay constant. The time decay constant τ is characteristic of a fluorophore type. As is known in the art, an equivalent way to express (1) is by replacing I with P, the probability of emission from a single fluorophore as a function of time. Furthermore, it can be determined that, when a fluorophore has a photon emission probability (fluorescence decay) of equation (1), the average time after excitation of photon emission from a fluorophore t̅ is equal to τ. That is, the average time t̅ for equation (1) may be determined as:

$$\overline{t} = \frac{{I}_{0} {\int }_{0}^{∞} {te}^{- \frac{t}{τ}} dt }{{I}_{0} {\int }_{0}^{∞} {e}^{- \frac{t}{τ}} dt} = \frac{τ ( t - τ ) {e}^{- \frac{t}{τ}} {\vert}_{0}^{∞}}{τ {e}^{- \frac{t}{τ}} {\vert}_{0}^{∞}} = \frac{{τ}^{2}}{τ} = τ \qquad ( 2 )$$

As such, the presence of a specific type fluorophore may be identified by determining whether the average time of photon detection by the fluorescence detector after a sufficiently large number of photons have been detected in response to a sequence of light pulses is substantially equal to τ. An embodiment of a circuit design to achieve this measurement is described herein.

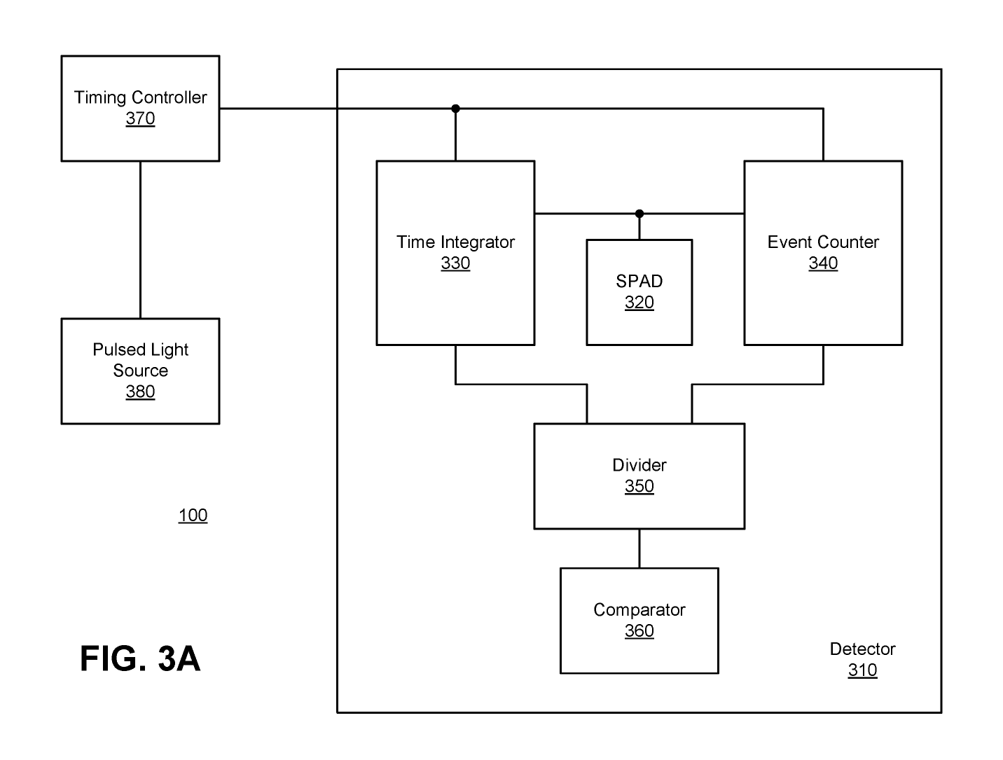

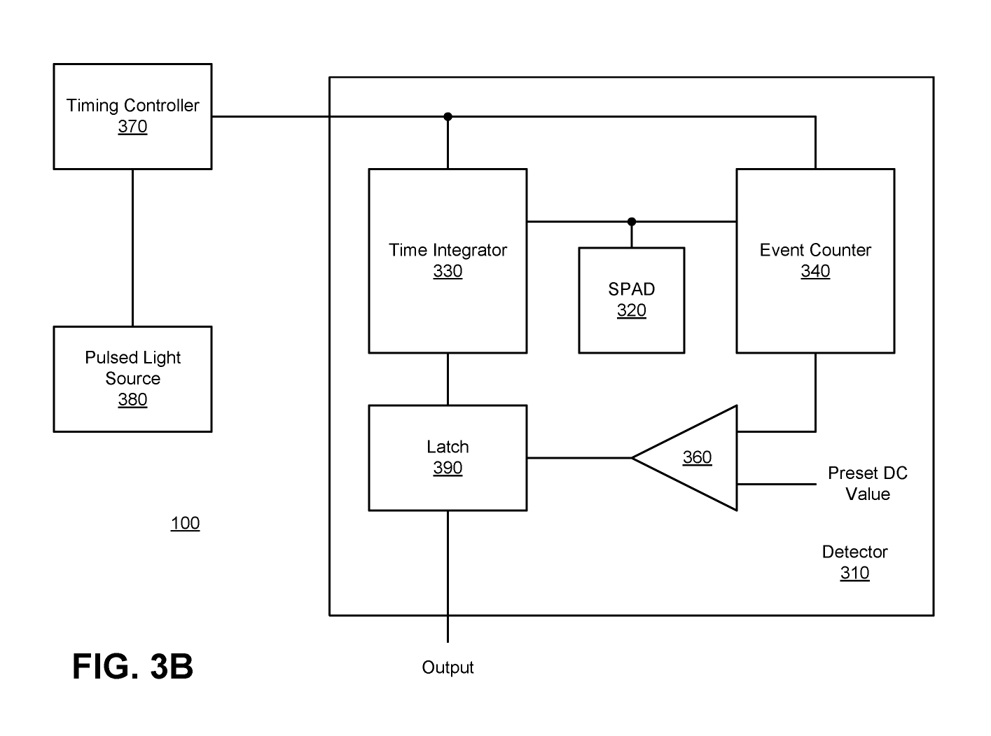

FIG. 3A illustrates a block diagram of a system for in vivo patient compliance, according to one embodiment. The sensor 100 includes a detector 310, a light source 380, and a timing controller 370. The timing controller 370 controls the activation of the detector 310 and the light source 380. In some embodiments, the connection between the timing controller 370 and the detector 310 has the same or substantially the same latency as the connection between the timing controller 370 and the light source 380. In other embodiments the latencies are not similar but are sufficiently stable over time, and the timing controller compensates for latency differences by delaying one of the control signals such that the SPAD is activated only after the light source has been triggered as described above. In some embodiments, the timing controller activates the light source and the detector 310 repeatedly for a preset number of times or cycles (e.g., 10,000 cycles). During each cycle, the pulsed light source 380 is activated to emit a short light pulse (e.g., as shown in FIG. 2), and the detector is activated for a preset time period (e.g., twice the length of the expected decay time constant τ). As such, the sensor 100 may account for the randomness of the fluorescent emissions by averaging the detections over a large sample size or may collect a sufficiently large number of photon arrival time so as to sufficiently reduce the error of the mean.

In some embodiments, activation of the pulsed light source 380 by the timing controller 370 includes 3 different levels to minimize power consumption: 1) a standby mode with low power consumption; 2) a sub-threshold mode with power consumption just below that sufficient to emit light from the pulsed light source; and 3) an active mode with power consumption sufficient to emit a rapid pulse of light from the pulsed light source. In order to reduce the power consumption by the device, the pulsed light source 380 is in a standby mode when not engaged in consecutive cycles of pulsing to excite fluorophores in the surrounding medium, is in sub-threshold mode during the active emission of a pulse train but in between pulse emissions, and in the active mode only during the pulsed emission of light.

The detector 310 includes a single-photon avalanche diode (SPAD) 320, a time integrator 330, an event counter 340, a divider 350, and a comparator 360. The SPAD 320 generates a signal in response to detecting a single photon and with a high time correlation to the physical absorption of the photon, also known as low jitter. The output of the SPAD 320 is coupled to the time integrator 330 and the event counter 340.

In some embodiments, the detector 310 includes multiple SPADs. Including multiple SPADs may increase the accuracy of the detector 310 by increasing the number of photons that can be detected at once. That is, if more than one photon reaches the detector 310, a detector that only has a single SPAD will only be able to detect the first photon that reaches the detector 310 and any subsequent photons will not be detected until the SPAD has been recharged. Consequently a bias will affect the statistical measure of arrival times, and a statistical error known as pile-up will result. Typically the pile-up problem can be prevented by ensuring that the probability of more than one photon impinging on the detector in response to a single laser pulse is very low, e.g., less than 1%. This increases the time for achieving a certain number of detections. Having multiple SPADs may reduce the latency due to the amount of time for charging a SPAD. That is, while a first SPAD is being discharged, a second SPAD may remain charged to allow the detector 310 to detect additional photons, thus reducing the amount of time to acquire a predefined number of photons in a non-light-flux-limited system.

Another system-level limitation in the design of SPAD-based systems is the trade-off between detection area and timing precision of the photon arrival-time-measurement. A similar trade-off exists between detection area and power consumption. Both of these trade-offs result from the larger capacitance of large-area junctions. Jitter increases in large-SPAD devices because the avalanche takes time to build sufficiently to be detected. In large junctions this time depends on where in the charge-depleted volume of the diode the photon is absorbed. This is less of a problem in small devices. Furthermore, the power consumption in SPAD detectors is dominated by the power required to recharge the junction following an avalanche. This power is determined by the product of the overbias of the junction (voltage beyond the breakdown voltage) and the junction capacitance. Larger SPADs will have higher capacitances and will therefore draw more power. Another drawback of large capacitance SPADs is that the total charge flowing during the avalanche scales as the product of the capacitance and overbias, and a correlated noise known as afterpulsing scales with this charge. However, a larger SPAD area is desirable in order to simplify focusing photons onto the active area.

One embodiment incorporates multiple SPADs, which are all connected to the same time integrator and event counter. A larger detection area is achieved because detection can occur in the total area of the SPADs. However, capacitance (together with power dissipation and noise) does not increase because a buffer (e.g., an inverter) is inserted between each of the diodes and the subsequent stages, thus buffering each junction's capacitance from that of the other junctions. Thus, if any of the SPADs undergoes an avalanche, a binary output of the buffer flips and the timing and counting gates are triggered.

In one implementation, the output of the SPAD inverters is connected to a NOR gate, such that when the first of the inverters changes output value, the output value of the NOR gate changes as well. In this implementation the inverters are designed for maximal matching. For example, non-minimum gate sizes are used. Also as an example, symmetrical layout and interconnect matching is implemented.

In one implementation the inverters are connected to a glitch detection circuit. At the beginning of a detection cycle, all the outputs of the inverters are at a given voltage. Once an avalanche discharges one of the SPADs, the corresponding inverter tries to change logic state but since the other inverters maintain their original state, the output node of the inverters undergoes a glitch, which is detected by the glitch detection circuit. In one implementation the glitch detection circuit has low timing jitter from detection to its output changing state. In one implementation the glitch detection circuitry latches the input such that once a glitch has been detected, the detection circuit changes state and maintains the new state until the detection circuit is reset. In one implementation, glitch detecting schemes such as those used in domino logic circuits are used to detect with low jitter the onset of avalanche of the first SPAD to discharge.

In subsequent paragraphs, SPAD and SPADs in the context of an array of SPADs connected to measurement circuitry are used interchangeably.

The time integrator 330 collects information regarding the time of arrival of a photon that triggered the SPAD 320 to generate a signal. In one embodiment, the time integrator stores an amount of charge that is correlated with the time interval between the start of the excitation light pulse and the arrival of the photon. For instance, when the photon arrives at the SPAD 320, the SPAD 320 outputs a signal to the time integrator 330. After receiving the output signal, the time integrator begins storing charge until the time controller outputs a signal that deactivates the time integrator 330. In this embodiment, the amount of charge stored is proportional to the complement of the time of arrival of each photon with respect to the duration of each cycle. In other embodiments, the SPAD 320 stores charge until an output signal is received from the SPAD 320. In some implementations, the time integrator can be disconnected from the current source, which produces the charge, which is integrated, such that, when inactive, leakage current and therefore power consumption is reduced.

In some embodiments, the time integrator aggregates the charge stored in each cycle and outputs a single value indicative of the aggregate amount of charge stored. In other embodiments, the time integrator outputs a value indicative of the amount of charge stored in each individual cycle and is discharged at the beginning of every cycle.

The event counter 340 collects information regarding the number of times the SPAD 320 produced an output since the beginning of a measurement sequence. For example, each time the SPAD 320 produces an output in response to a photon, the event counter 340 stores a preset amount of charge. As such, the event counter 340 produces an output that is indicative of the number of times the SPAD 320 produced an output. In other words, the event counter 340 produces an output that is indicative of the number of cycles in which a photon reached the SPAD 320. In some embodiments, the event counter can be disconnected from the current source, such that, when inactive, leakage current and therefore power consumption is reduced.

The divider 350 receives the amount of charge stored by the time integrator 330 and the amount of charge stored by the event counter 340, and the divider 350 divides the two amounts. In one embodiment, the amount of voltage stored by the time integrator 330 and the amount of charge stored by the event counter 340 is received as voltage values that are proportional to the respective amounts of charge. In one embodiment, the divider 350 digitizes the voltages that are indicative of the amount of charge stored by the time integrator 330 and the event counter 340, and calculates the ratio using digital circuitry. In another embodiment, the ratio between the voltage indicative of the charge stored by the time integrator 330 and the voltage indicative of the charge stored by the event counter 340 is determined using a Gilbert Multiplier.

The comparator 360 receives the ratio determined by the divider 350 and compares the ratio to an expected value. For instance, the comparator 360 compares the ratio to an expected decay time constant τ for a specific fluorophore. In some embodiments, the comparator outputs a binary value that indicates whether presence of the target fluorophores was detected.

FIG. 3B illustrates a block diagram of a system for in vivo patient compliance, according to another embodiment. In the embodiment of FIG. 3B, no charge divider is used. Instead, a preset dc value is compared in a comparator 360 with the voltage of the event counter 340. Once the event counter 340 reaches the preset DC value, the comparator's output is switched, the timing integrator's 330 voltage is read out (directly, buffered, or digitized), and the whole detector circuit is reset (i.e. all SPADs 320 are recharged, and the time integrator 330 and event counter 340 are reset). In this case no division is necessary because the number of detection events is known and represents a constant. In one implementation, upon reaching the preset number of events, the integrated voltage representing the sum of times is latched using latch 390 and the detector circuit is discharged so that the SPADs are inactive and the measurement circuitry is inactive.

The pulsed light source 380 includes one or more light emitting elements that can be turned on and off significantly faster than the decay time constant of the fluorophore being detected. In some embodiments, the pulsed light source 380 includes one or more light emitting diodes (LEDs) or vertical-cavity surface-emitting lasers (VCSELs).

In some embodiments, the pulsed light source 380 includes an optical filter.

In some embodiments, an optical filter, which is in the light path of the excitation light path but not in the light path of the imaged light path attenuates the light that is outside of the fluorescence band of the fluorophores to reduce the amount of background light being detected.

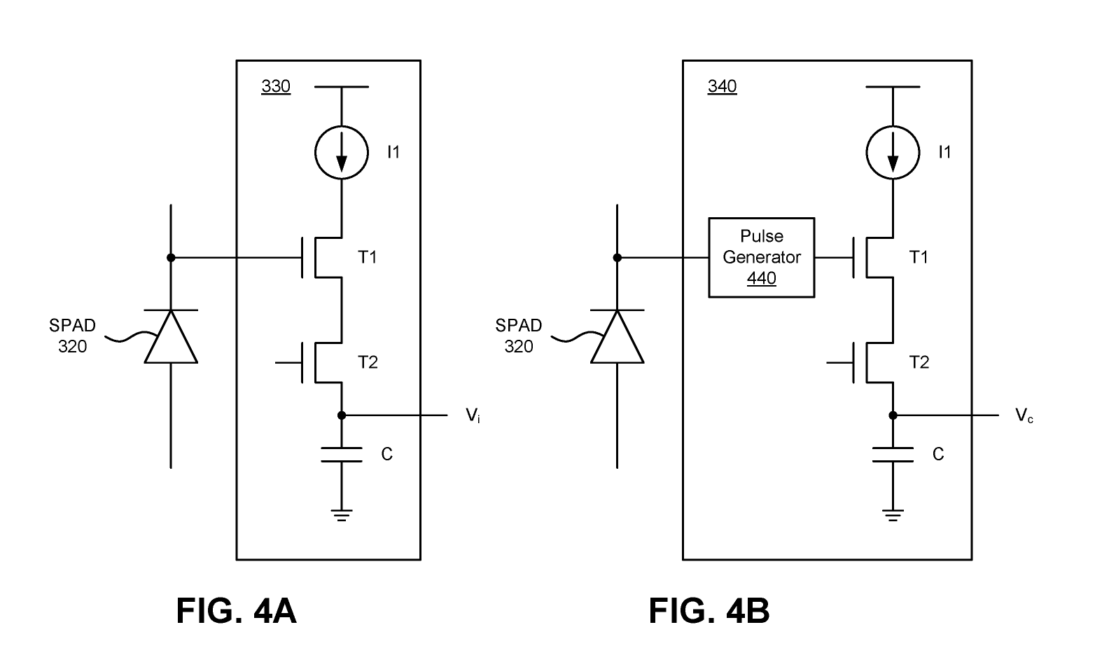

FIG. 4A illustrates a circuit diagram of the time integrator of FIG. 3A, according to one embodiment. The time integrator 330 includes a first transistor T1 that is controlled by the output of the SPAD 320. That is, the first transistor T1 turns on or off based on a signal received from the SPAD 320. The time integrator 330 further includes a second transistor T2 that is connected in series to the first transistor T1. The second transistor is controlled by the timing controller 370. When both the first transistor T1 and the second transistor T2 are on, a current generated by current source I1 charges a capacitor C. If the current generated by current source I1 is constant, the amount of charge stored in capacitor C is proportional to the amount of time both the first transistor T1 and the second transistor T2 were concurrently on.

During each cycle, the timing controller 370 turns on the second transistor T2 at the beginning of the cycle, and turns off the second transistor T2 after a preset amount of time (e.g., twice the decay time constant of the target fluorophore). As such, the amount of charge stored in the capacitor during each cycle is as follows:

Qᵢ=I₁Δt=I₁(t_{end}−tᵢ) (3)

Where Qᵢ is the amount of charge stored in capacitor C during one cycle, I₁ is the current generated by current source I₁, t_{end} is the duration of time during which the second transistor is on (i.e., the duration of the sensing period), and tᵢ is the time of arrival of the photon that caused the SPAD 320 to generate an output signal. Moreover, after a preset number of cycles (e.g., 10,000 cycles), the total amount of charge (Q_{iT}) stored in capacitor C is as follows:

$${Q}_{iT} = {\sum }_{i = 1}^{n} {I}_{1} ( {t}_{end} - {t}_{i} ) \qquad ( 4 )$$

Where n is the number of times a photon reached the SPAD 320. Without limiting the generality of the above description, additional circuit elements may be added or altered in the time integrator circuit without altering its basic functionality, for example, in order to reduce quiescent or power-down power consumption, to reduce the effect of process, temperature and voltage fluctuations, to reduce glitches in the power supplies (for example by adding capacitors), etc.

In some embodiments, the time integrator 330 outputs a voltage Vᵢ that is proportional to the amount of charge stored in the capacitor C. In particular, the output voltage Vᵢ looks as follows:

$${V}_{i} = \frac{{Q}_{iT}}{C} = \frac{{I}_{1}}{C} {\sum }_{i = 1}^{n} ( {t}_{end} - {t}_{i} ) = \frac{{I}_{1}}{C} {nt}_{end} - \frac{{I}_{1}}{C} {\sum }_{i = 1}^{n} {t}_{i} \qquad ( 5 )$$

FIG. 4B illustrates a circuit diagram of the event counter of FIG. 3A, according to one embodiment. FIG. 4B includes a circuit similar to the time integrator 330 with an additional pulse generator 440 at the gate of the first transistor T1. When the SPAD 320 generates an output indicative of a photon reaching the SPAD 320, the pulse generator generates a pulse with a preset shape (e.g., with a preset amplitude and duration). As such, during each cycle where a photon induces a detectable avalanche in the SPAD 320, the amount of charge stored in capacitor C of event counter 340 looks as follows:

Q_{c}=I₁(tₚ) (6)

Where Q_{c} is the amount of charge stored in capacitor C during one cycle, I₁ is the current generated by current source I1, tₚ is the duration of time of the pulse generated by pulse generator 440. Moreover, after a preset number of cycles (e.g., 10,000 cycles), the total amount of charge (Q_{T}) stored in capacitor C is as follows:

$${Q}_{cT} = {\sum }_{i = 1}^{n} {I}_{1} ( {t}_{p} ) = {nI}_{1} {t}_{p} \qquad ( 7 )$$

Where n is the number of times a photon reached the SPAD 320. Since both the time integrator and the event counter are controlled by the same SPAD, the value of n is the same for both Q_{iT} and Q_{cT}.

In some embodiments, the event counter 340 outputs a voltage V_{c} that is proportional to the amount of charge stored in the capacitor C. In particular, the output voltage V_{c} looks as follows:

$${V}_{c} = \frac{{Q}_{cT}}{C} = \frac{{I}_{1}}{C} {\sum }_{i = 1}^{n} ( {t}_{p} ) = \frac{{I}_{1}}{C} {nt}_{p} \qquad ( 8 )$$

Thus, the ratio between the output voltage of the time integrator 330 and the output voltage of the event counter 340 is:

$$\frac{{V}_{i}}{{V}_{c}} = \frac{{Q}_{iT}}{{Q}_{cT}} = \frac{{Σ}_{i = 1}^{n} ( {t}_{end} - {t}_{i} )}{{nt}_{p}} = \frac{1}{{t}_{p}} ( {t}_{end} - \frac{1}{n} {\sum }_{i = 1}^{n} {t}_{i} ) \qquad ( 9 )$$

### Where

$$\frac{1}{n} {Σ}_{i = 1}^{n} {t}_{i}$$

is the average time or a photon reaching the SPAD 320. Thus, to determine whether the fluorescence was generated by the target fluorophore, the voltage ratio may be compared to:

$$\frac{1}{{t}_{p}} ( {t}_{end} - τ ) \qquad ( 10 )$$

Moreover, if the t_{end} is chosen to be twice the decay time constant of the target fluorophore, then the ratio may be compared to:

$$\frac{1}{{t}_{p}} ( 2 τ - τ ) = \frac{τ}{{t}_{p}} \qquad ( 11 )$$

Without limiting the generality of the above description, additional circuit elements may be added or altered in the event counter circuit without altering its basic functionality, for example, in order to reduce quiescent or power-down power consumption, to reduce the effect of process, temperature and voltage fluctuations, to reduce glitches in the power supplies (for example by adding capacitors), etc.

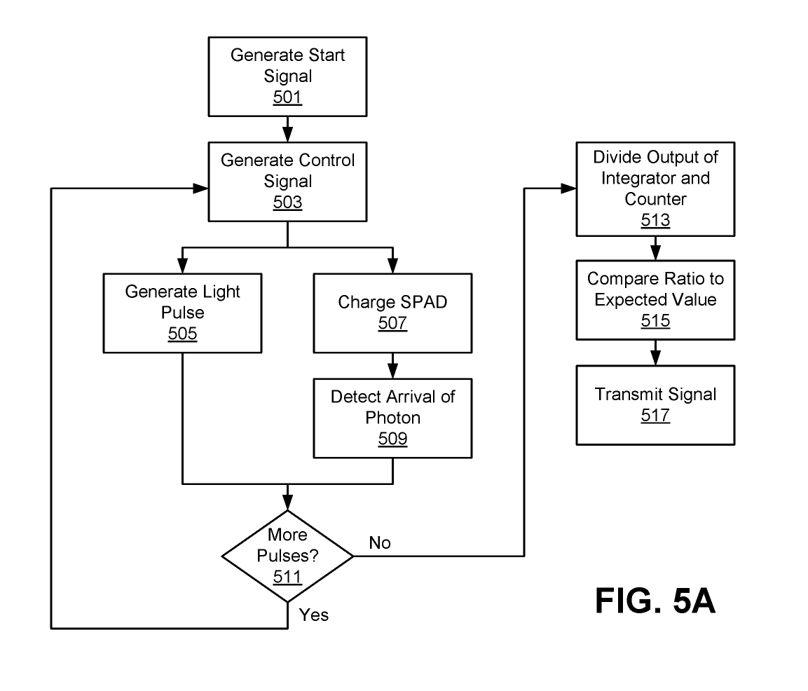

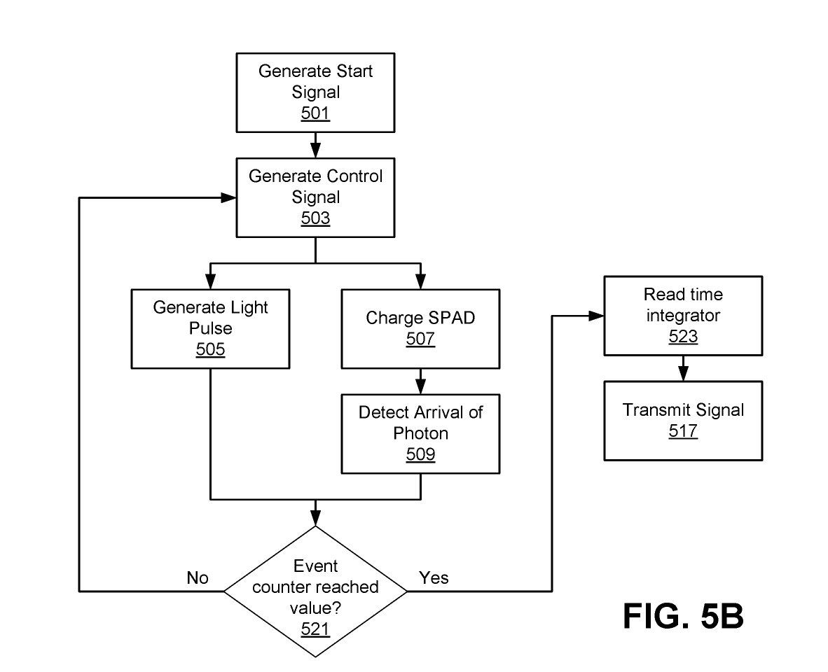

FIG. 5A illustrates a flow diagram of a process for in vivo patient compliance determination, according to one embodiment. A start scan pulse is generated 501. In one embodiment, the signal is generated periodically, for example once every hour. In one embodiment the 50 consecutive sequences of pulses, each 100,000 pulses in length may be generated once every 3 hours. The signal can be generated by an interconnected implanted device, such as a pacemaker or other device implanted in the body of the patient. The signal can be generated by an interconnected ingested device, such as a camera pill or other device ingested or otherwise internalized in the body of the patient. In other implementations, the signal is generated outside the body of the patient and is transmitted to the sensor 100 wirelessly.

A control signal is generated 503 to enable the pulsed light source 380 and the detector 310. Based on the control signal, the pulsed light source 380 initially powers to a subthreshold, “Ready” state. The pulsed light source 380 subsequently generates 505 one or more light pulses. In some embodiments, the generated light pulse(s) propagate through a lens such that the light pulse(s) illuminate a wide band of angles.

Based on the control signal, the SPAD 320 is charged 507 beyond breakdown to allow the SPAD 320 to detect a single photon reaching the SPAD 320. In some embodiments, the SPAD 320 is charged in response to a delayed version of the control signal. For example, the control signal is delayed by a duration of the light pulse generated by the pulsed light source 308. In some embodiments, the control signal is delayed until the power of the light pulse falls to 1% of the peak instantaneous power.

The SPAD 320 is left charged for a preset time (active time). In some embodiments, the active time is on the order of 2 times the time decay constant of the target fluorophore. After the active time, a discharge signal discharges the voltage across the SPAD 320 to below the breakdown voltage of the SPAD 320. If a photon is absorbed in the pn junction of the SPAD during the active time, an avalanche breakdown builds up in the SPAD 320.

The arrival of a photon to the SPAD 320 is detected 509. After the avalanche breakdown builds up in the SPAD 320 in response to the pn junction of the SPAD absorbing a photon, transistors T1 of time integrator 330 and event counter 340 are turned on. An amount of charge based on the time the photon was absorbed by SPAD 320 is stored in capacitor C of time integrator 330. Additionally, a preset amount of charge is stored in capacitor C of event counter 340.

After the detection period ends, a determination 511 is made whether all the light pulses have been generated. For instance, a counter may keep count of the number of pulses generated. If additional pulses are to be generated, the process returns to step 503.

If all the light pulses have been generated, the output of the time integrator 330 and the output of the event counter 340 are divided 515. The determined ratio between the output of the time integrator 330 and the output of the event counter 340 is compared to an expected value or an expected range using comparator 360.

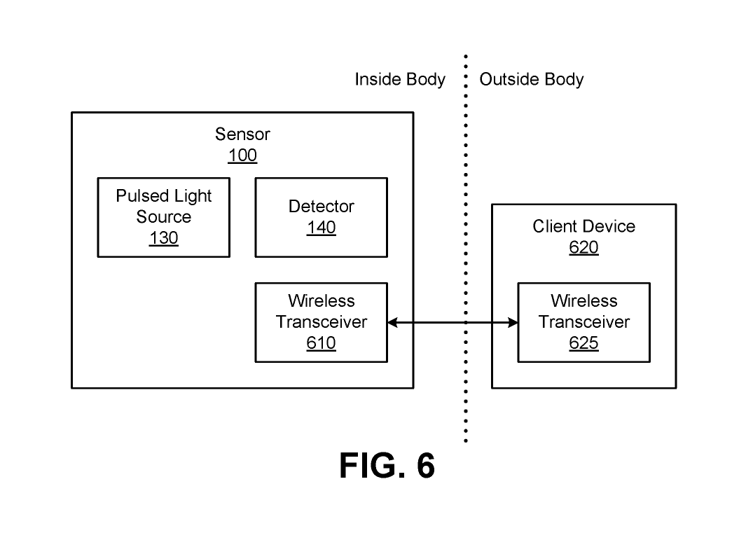

In some embodiments, the output of the comparator 360 is transmitted to a client device that is outside of the patient's body. FIG. 6 illustrates a block diagram of an environment of a system for in vivo patient compliance communicating with an external client device. In addition to the components described hereinabove, the sensor 100 includes a wireless transceiver 610 that communicates with a wireless transceiver 625 of a client device 620 that is outside the body of the patient. As such, the measurements obtained by sensor 100 can be transmitted to the client device 620 for further processing. In some embodiments, as shown in FIG. 1B, the system for in vivo patient compliance communicates with the client device 620 using a wireless interface of second system that is included in the same enclosure. The system for in vivo patient compliance may have a wired connection to the second system. The system for in vivo patient compliance then transmits the data to be transmitted to the client device, and to the second system via the wired connection.

In one embodiment, a software program running in the client device 620 records the times of detection and generates a report. Classification of detection events as false positives or false negatives may be carried out based on the duration of detected events. For example, spurious detections may be classified as false. In one implementation, the report compares the recorded times of detection of the presence of the fluorophores and compares the detections with expected detection times based on a preset schedule. If detection events do not match with expected times, the software generates an alert or notification. The notification and/or report can be delivered to a client device (e.g., mobile phone), to the patient, to a physician or health care provider of the patient, to a family member or friend of the patient, etc.

FIG. 5B illustrates a flow diagram of a process for in vivo patient compliance determination, according to another embodiment. Similarly to the embodiment of FIG. 5A, a scan pulse is generated 501. After the start signal is generated, a control signal is generated 503 to enable the pulsed light source 380 and the detector 310. Based on the control signal, the pulsed light source 380 generates 505 one or more light pulses, and the SPAD 320 is charged 507 beyond breakdown to allow the SPAD 320 to detect a single photon reaching the SPAD 320. The SPAD 320 is left charged for a preset time (active time). If a photon is absorbed in the pn junction of the SPAD during the active time, an avalanche breakdown builds up in the SPAD 320.

The arrival of a photon to the SPAD 320 is detected 509. After the avalanche breakdown builds up in the SPAD 320 in response to the pn junction of the SPAD absorbing a photon, transistors T1 of time integrator 330 and event counter 340 are turned on. An amount of charge based on the time the photon was absorbed by SPAD 320 is stored in capacitor C of time integrator 330. Additionally, a preset amount of charge is stored in capacitor C of event counter 340.

A determination is made whether the output of the event counter 340 reached a preset value. For instance, the output of the event counter 340 is compared to a preset value using a comparator. If the output of the event counter has not reached the preset value, the process returns to step 503.

If the output of the event counter 340 has reached the preset value, the output of the time integrator 330 is read 523. In some embodiments, the output of the time integrator 330 is compared to an expected value and the output of the comparison is transmitted 517 to a client device. In other embodiments, the output of the time integrator 330 is simply transmitted 517 to the client device for further processing.

Thus, the invention provides ongoing in vivo or internal monitoring of patient medication compliance over time. It conveniently allows health care providers and others associated with a patient to determine whether the patient is taking or not taking medication, and whether the medication is being taken at the appropriate times. It does this via a system that includes an in-vivo, ultra-low-power, highly-specific, non-imaging monitoring device that monitors for the presence or absence of a specific fluorophore. The system solves various technical problems associated with internal fluorescence detection. For example, there is a limited number of fluorescence molecules that can be incorporated into a pill or other medication. In addition, the amount of fluorescently labeled medication in the blood will be small, and the medication will dispersed in the blood. Thus, there will be a low concentration of fluorescent marker in the body to be detected by the system. In addition, there is a certain amount of fluorescence occurring in tissue of the body naturally, creating background noise that makes it difficult to correctly detect fluorescence originating from the medication. By using both a spectral filter (e.g., excitation and fluorescence emission filters) as well as a fluorescence-lifetime filter, the system maximizes signal to background in an optically noisy, low-signal environment. The system provides regular reporting of patient medication compliance over a period of time without discomfort to or effort by the patient.

The system can be used for monitoring of anything within the body that includes a fluorescent marker, including any substance taken into the body or any labeled internal bodily component. In some embodiments, the system comprises any type of single photon detector, either a single detector or an array of detectors, and includes a circuit to measure the total time and the number of detected events, and their ratio. In some embodiments, an array of detectors is preferred to facilitate detection of multiple photons over a given time after an excitation pulse. In some embodiments, the power of the light pulse or the concentration of fluorophore anticipated is low enough such that only a single detector can provide sufficient information and is more power efficient. The detector can be mounted on, incorporated into, or associated with any type of implantable or ingestible item. The detector can also be included on or within a capsule or other ingested by the patient by the patient or otherwise inserted into the patient in some manner. The detector can also be implanted within or near a particular structure in the body, such as a tumor. The system can be used for medication compliance monitoring, or any other type of monitoring or detection associated with medication or ingested materials.

### Additional Configuration Considerations

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Certain embodiments are described herein as including logic or a number of components, modules, or mechanisms. Modules may constitute either software modules (e.g., code embodied on a machine-readable medium or in a transmission signal) or hardware modules. A hardware module is tangible unit capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

The one or more processors may also operate to support performance of the relevant operations in a “cloud computing” environment or as a “software as a service” (SaaS). For example, at least some of the operations may be performed by a group of computers (as examples of machines including processors), these operations being accessible via a network (e.g., the Internet) and via one or more appropriate interfaces (e.g., application program interfaces (APIs).)

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Some portions of this specification are presented in terms of algorithms or symbolic representations of operations on data stored as bits or binary digital signals within a machine memory (e.g., a computer memory). These algorithms or symbolic representations are examples of techniques used by those of ordinary skill in the data processing arts to convey the substance of their work to others skilled in the art. As used herein, an “algorithm” is a self-consistent sequence of operations or similar processing leading to a desired result. In this context, algorithms and operations involve physical manipulation of physical quantities. Typically, but not necessarily, such quantities may take the form of electrical, magnetic, or optical signals capable of being stored, accessed, transferred, combined, compared, or otherwise manipulated by a machine. It is convenient at times, principally for reasons of common usage, to refer to such signals using words such as “data,” “content,” “bits,” “values,” “elements,” “symbols,” “characters,” “terms,” “numbers,” “numerals,” or the like. These words, however, are merely convenient labels and are to be associated with appropriate physical quantities.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the term “implantable” refers to any device that is inserted into or fixed within a person's body. This can include devices that are fixed into place, such as a pacemaker, but can also include devices that move within the body of a patient, such as a pill camera. Means of insertion into a patient's body of an implantable or implanted device can be, e.g., through surgery, through ingestion, or through other mechanisms of insertion.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the invention. This description should be read to include one or at least one and the singular also includes the plural unless it is obvious that it is meant otherwise.

Upon reading this disclosure, those of skill in the art will appreciate still additional alternative structural and functional designs for a system and a process for in vivo patient compliance monitoring through the disclosed principles herein. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation, and details of the method and apparatus disclosed herein without departing from the spirit and scope defined in the appended claims.

What is claimed is:

1. An in vivo patient compliance apparatus comprising:
a timing controller;
a pulsed light source coupled to the timing controller; and
a detector, the detector comprising:
a single-photon avalanche diode (SPAD),
a time integrator coupled to the SPAD and the timing controller, the time integrator configured to store charge in response to receiving a signal from the SPAD, and configured to stop storing charge in response to receiving a signal from the timing controller, and
an event counter coupled to the SPAD, the event counter configured to store a preset amount of charge in response to receiving a signal from the SPAD.

2. The in vivo patient compliance apparatus of claim 1, wherein the time integrator comprises:
a first transistor controlled by the SPAD;
a second transistor coupled to the first transistor, the second transistor controlled by the timing controller; and
a capacitor coupled to the second transistor, the capacitor configured to store charge in response to the first transistor and the second transistor being turned on.

3. The in vivo patient compliance apparatus of claim 1, wherein the event counter comprises:
a pulse generator coupled to the SPAD, the pulse generator configured to generate a pulse with a preset shape;
a first transistor coupled to an output of the pulse generator, the first transistor controlled by the pulse generator, the first transistor configured to turn on when the output of the pulse generator is above a threshold level;
a second transistor coupled to the first transistor, the second transistor controlled by the timing controller; and
a capacitor coupled to the second transistor, the capacitor configured to store charge in response to the first transistor and the second transistor being turned on.

4. The in vivo patient compliance apparatus of claim 1, further comprising:
a divider coupled to the time integrator and the event counter, the divider configured to divide an output of the time integrator and an output of the event counter; and
a comparator coupled to the divider, the comparator configured to compare an output of the divider to a predetermined value.

5. The in vivo patient compliance apparatus of claim 4, wherein the predetermined value is a decay time constant of a fluorophore.

6. The in vivo patient compliance apparatus of claim 1, further comprising:
a comparator coupled to an output of the event counter, the comparator configured to compare the output of the event counter to a preset value; and
a latch coupled to an output of the comparator and an output of the time integrator, the latch configured to store the output of the time integrator in response to receiving a signal from the comparator.

7. The in vivo patient compliance apparatus of claim 1, wherein the comparator comprises:
a differential amplifier configured to output a first voltage level in response to the output of the event counter being greater than the preset value, and a second voltage level in response to the output of the event counter being lower than the preset value.

8. The in vivo patient compliance apparatus of claim 1, wherein the in vivo patient compliance apparatus is integrated in a pacemaker of a patient, and further comprising:
a wireless interface for communicating to a wireless receiver outside the body of the patient.

9. The in vivo patient compliance apparatus of claim 1, wherein the timing controller is configure to generate a sequence of control signals for controlling the pulsed light source and charging the SPAD.

10. The in vivo patient compliance apparatus of claim 9, wherein the sequence of control signals include at least 10,000 pulses.

11. The in vivo patient compliance apparatus of claim 9, wherein the SPAD is charged for at least twice the decay time constant of a fluorophore.

12. A method comprising:
initializing a time integrator and an event counter;
for each of a plurality of cycles:
generating a light pulse,
charging a single-photon avalanche diode (SPAD),
detecting an arrival of a photon using the SPAD, and
responsive to detecting the arrival of the photon:
starting the time integrator, and
increasing a value of the event counter; and
comparing a value of the time integrator and the event counter to identify the presence of a fluorophore.

13. The method of claim 12, wherein comparing the value of the time integrator and the event counter comprises:
determining a ratio of an output of the time integrator and an output of the event counter, and
comparing the determined ratio to an expected value.

14. The method of claim 12, further comprising:
for each of the plurality of cycles, responsive to the event counter reaching a preset value, storing a value of the time integrator.

15. The method of claim 14, further comprising:
responsive to the stored value of the time integrator having an expected value, identifying the presence of the fluorophore.

16. The method of claim 12, wherein increasing a value of the event counter comprises:
increasing an amount of charge stored in capacitor of the event counter by a preset amount of charge.

17. The method of claim 12, wherein generated light pulse has a predetermined intensity and duration.

18. The method of claim 17, wherein the predetermined intensity and duration is based on the fluorophore to be detected.

19. The method of claim 12, wherein the identification of the presence of the fluorophore is performed inside a body of a patient, and further comprising:
transmitting a signal indicating the presence of the fluorophore wirelessly to a receiver outside a body of the patient.

20. The method of claim 12, wherein the plurality of cycles includes at least 10,000 cycles.
